(19) United States
(12) Patent Application Publication
(10) Pub. No.: US 2017/0311662 A1
(43) Pub. Date: Nov. 2, 2017

(54) Post-Operative Lower Body Garment

(71) Applicants: Deysi Piña, Miami, FL (US); Alicia Glatzer, Palmetto Bay, FL (US)

(72) Inventors: Deysi Piña, Miami, FL (US); Alicia Glatzer, Palmetto Bay, FL (US)

(21) Appl. No.: 15/583,950

(22) Filed: May 1, 2017

Related U.S. Application Data
(60) Provisional application No. 62/329,471, filed on Apr. 29, 2016.

(51) Int. Cl. A41D 13/12; A61M 27/00; A61M 5/142; A41F 9/02; A41D 27/20

(52) U.S. Cl. CPC A41D 13/1254; A41F 9/025; A41D 27/205; A61M 5/142; A61M 27/00; A41D 2300/33; A41D 2300/322; A61M 2209/088

(57) ABSTRACT

A post-operative lower body garment has a plurality of inner pockets connected to the interior of the waist portion of the garment. The inner pockets are configured to receive a medical device such as a Jackson-Pratt drain or an insulin pump, and have multiple holes for accommodating any tubes connecting the medical device to the user's body. A lower body portion of the garment comprising pant legs may have invisible zippers integrated into the lower portion of the pant legs for easy access to the user's legs without requiring the user to remove the garment.

The current application claims a priority to the U.S. Provisional Patent application Ser. No. 62/329,471 filed on Apr. 29, 2017. The current application is filed on May 1, 2017, whereas Apr. 29, 2017 and Apr. 30, 2017 were on a weekend.

### FIELD OF THE INVENTION

The present invention relates generally to clothing. Specifically, the present invention relates to a garment for patients that have undergone a surgical procedure from the abdomen to the lower extremities.

### BACKGROUND OF THE INVENTION

Thousands, if not millions of individuals throughout the world undergo surgeries every day. Certain surgeries, such as bilateral mastectomies require the use of postoperative devices which may include Jackson-Pratt (JP) drain bulbs or similar drainage units that connect to the body in order to remove fluid from the point of surgery. Drain bulbs are often taped to the patient's skin, while tubing is used to connect the bulb to the wearer's body. Both are intrusive, unsightly and uncomfortable for the patient. The removal of tape from a user's skin is often painful and can also irritate the point of connection between the tube and the surgical site. As a result, inventors have developed various clothing articles to hold drain bulbs. Unfortunately, the prior inventions are often uncomfortable to wear as they exert pressure on the patient's mid-section and lack flexibility with positioning of the drainage tubing. Such garments must often be removed, sometimes constantly, in order to allow individuals to perform necessary adjustments of their postoperative devices. This is not only time consuming and painful when recovering but may prove to be embarrassing depending on the setting.

As such, it is therefore an objective of the present invention to introduce new a new lower body worn post operative garment. The present invention is generally worn by male or female patients, as well as children who have undergone surgery in and around the abdomen, being particularly useful as it utilizes a loose-fitting waistband with a drawstring to alleviate pressure on the waist and abdomen. Moreover, the present invention can be worn by any and all age groups and sexes throughout their post-operative or procedural rehabilitation periods. The garment utilizes a plurality of inner pockets which can hold drainage reservoirs, such as JP drain bulbs and insulin pumps. The pockets can furthermore hold cell phones and other small devices. The pants include the use of invisible zippers along the inner and outer side seams of the left leg sleeve and right leg sleeve. The invisible zippers begin at the bottom of each pant leg, extending up towards the bottom or above the knee. These features significantly benefit patients who wear urinary catheters, as they provide a convenient means to reach the drainage bag without the need to remove the pants. Similarly, having zippers on the inner and outer seams of the pants can facilitate someone who is wearing a cast. Amputees would also benefit as they could easily put on their prosthesis simply by unzipping both sides of the pant leg. Doctors can easily examine the healing of a patient's fracture, wound, incision and/or medical device by unzipping said zippers. The use of invisible zippers further allows for discrete use of the pants while maintaining the wearer's dignity. Overall, the present invention is practical, comfortable, simple, safe, and effective.

### BRIEF DESCRIPTION OF THE DRAWINGS

FIG. 1 is an elevated front-side view of the garment;

FIG. 2 is an elevated rear-side view of the garment thereof;

FIG. 3 is an elevated top-side view of the garment thereof,

FIG. 4 is a front-perspective view of the garment thereof;

FIG. 5 is an environmental view of the garment being worn by a postoperative patient;

FIG. 6 is an elevated front-side view of a shorts embodiment of the garment; and

FIG. 7 is an elevated front-side view of a skirt embodiment of the garment.

### DETAILED DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention.

The present invention is a lower body worn post-operative garment that can be worn and utilized for patients having any lower extremity devices including, but not limited to, casts, braces, straps, external fixation devices, hinged braces, knee supports and braces, prosthetics and any other type of lower extremity devices including post-operative devices. The present invention may be generally worn by patients who have undergone surgery in and around the abdomen, being particularly useful as it utilizes a loose-fitting waistband with a drawstring to alleviate pressure on the waist and abdomen. Due to the versatility of the present invention, the garment can be utilized and worn by any patient requiring either a left or right lower extremity cast or any other medical device. Moreover, the present invention can be worn by any and all age groups and sexes throughout their post-operative or procedural rehabilitation periods. The present invention may be worn by a patient immediately following an operation or other procedure, and may also be worn for extended periods of recovery such as a rehabilitation period. The garment utilizes a plurality of inner pockets which can hold drainage reservoirs, such as Jackson-Pratt (JP) drain bulbs and insulin pumps; however, cell phones and other small objects may be held as well. The interior pockets provide the user with privacy and convenience in concealing and securing their post-operative device, such as a JP drain, insulin pump or other device or devices.

Furthermore, the pants include the use of invisible zippers along the inner and outer seams of both the left leg sleeve and right leg sleeve. This allows accessibility, convenience, flexibility and comfort for the patient as well as ease of access of their medical providers and caregivers. The invisible zippers begin at the bottom of each pant leg and extend up towards the bottom of the knee or above the knee. Before explaining at least one embodiment of the present invention in detail, it is to be understood that the device is not limited in its application to the details of the components and arrangements as described or illustrated. The invention is capable of numerous other embodiments and utilizations all carried out in various ways. It is also to be understood that the phrasing and terminology employed herein are for the purpose description and should not be regarded as limiting. As such, the present invention is primarily worn on the lower body of post-operative patients and patients having undergone any and all bilateral or unilateral lower extremity procedures or surgeries; however, the garment may be applied to and utilized in many other settings, situations, and scenarios.

In the preferred embodiment, the present invention is a lower body garment—specifically, a pair of pants. In alternate embodiments, the present invention may be constructed as other lower body garments, including, but not limited to, shorts, or a skirt. The garment may be made primarily from one or more fabric materials which are well known by those skilled in the art, including, but not being limited to denim, silk, cotton, and more. The garment is generally intended for both male and female post-operative patients; however, the garment may also be worn by children, teenagers, elderly persons or any other gender and age group.

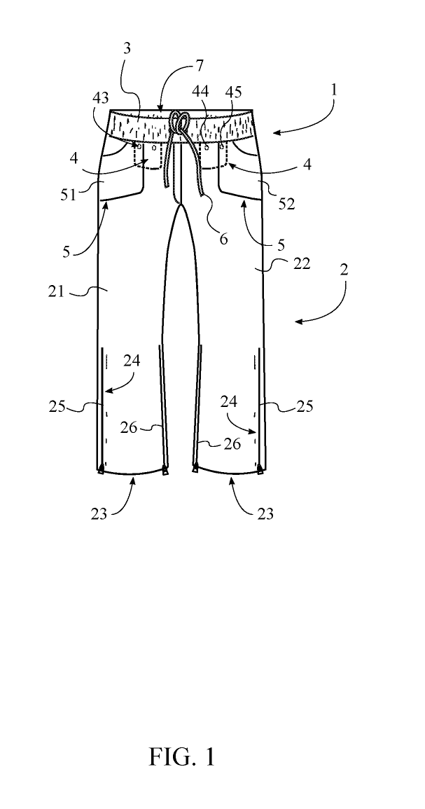

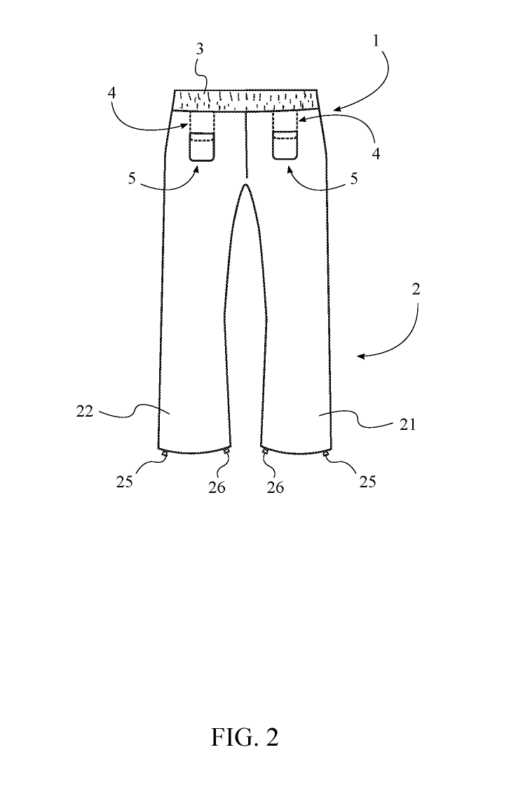

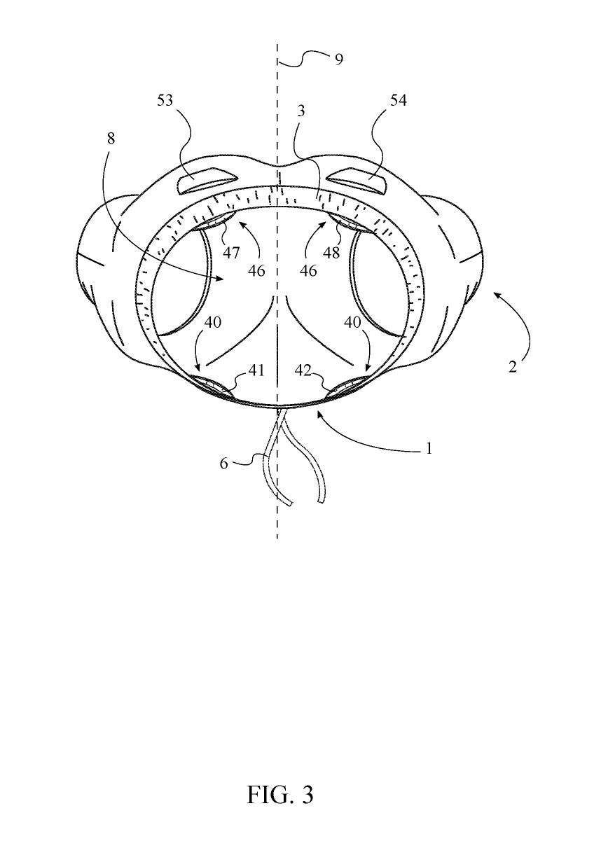

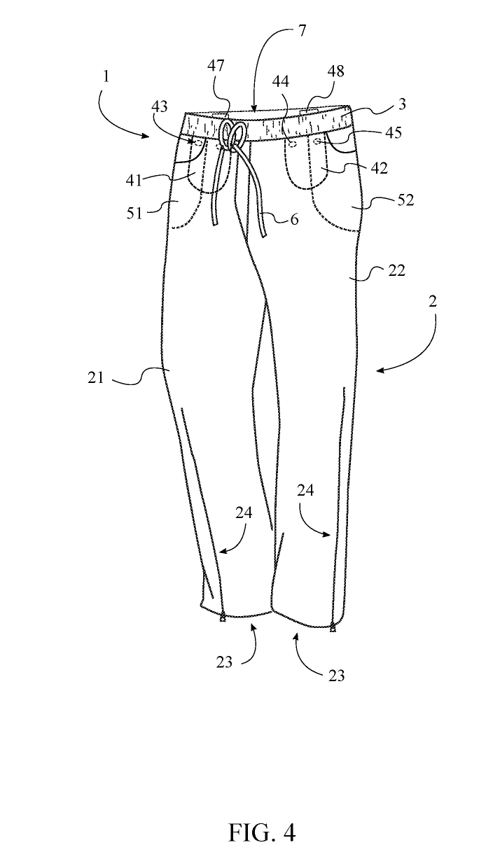

Referring to FIG. 1-4, the present invention generally comprises a waist portion 1 and a lower body portion 2. The waist portion 1 comprises a waistband 3, a plurality of inner pockets 4, and a plurality of outer pockets 5. In the preferred embodiment, the waistband 3 is made of elastic material. Furthermore, the waistband 3 comprises a drawstring 6 positioned within the waistband 3 such that the waistband 3 is configured to close an opening 7 of the waistband 3 so that the user can tighten the waistband 3 around their waist to a desired comfort level.

The lower body portion 2 is connected to the waist portion 1 opposite the waistband 3, wherein the waistband 3 delineates the opening 7 of the waist portion 1. The opening 7 traverses into an interior cavity 8 of the waist portion 1, such that in order to use the present invention, a user inserts their lower body through the opening 7 into the interior cavity 8. Each of the plurality of inner pockets 4 is interiorly connected within the interior cavity 8 of the waist portion 1, preferably between the waistband 3 and the lower body portion 2. In one embodiment, the plurality of inner pockets 4 is connected directly to the waistband 3 such that the plurality of inner pockets 4 essentially hangs from the waistband 3. In another embodiment, the plurality of inner pockets 4 is affixed to the interior of the waist portion 1. The plurality of outer pockets 5 is exteriorly connected to the waist portion 1 between the waistband 3 and the lower body portion 2. The plurality of outer pockets 5 may be considered to correspond to any configuration of pockets found in typical pants or similar lower body garments.

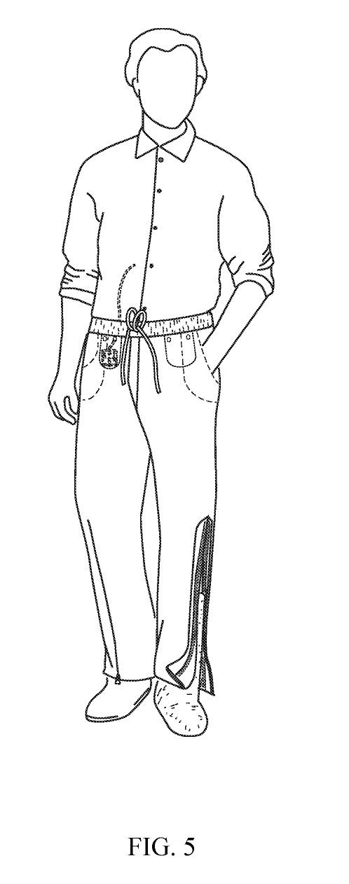

In the preferred embodiment of the present invention, the plurality of inner pockets 4 comprises a plurality of front inner pockets 40 and a plurality of rear inner pockets 46. It is contemplated, however, that alternate embodiments of the present invention may comprise only the plurality of front inner pockets 40, or the plurality of rear inner pockets 46. It is furthermore contemplated that some embodiments of the present invention may comprise a single front pocket, or a single rear pocket, or any combination of the aforementioned variations. The plurality of front inner pockets 40 and the plurality of rear inner pockets 46 are positioned longitudinally opposite each other within the waist portion 1, wherein the longitudinal direction should be understood to be horizontally along the sagittal plane of a human body when a user is wearing the present invention. In one embodiment of the present invention, at least one of the plurality of inner pockets 4 is configured to receive a post-operative drain reservoir. More specifically, in one embodiment of the present invention, at least one of the plurality of inner pockets 4 is configured to receive a Jackson-Pratt drain reservoir. In one embodiment of the present invention, at least one of the plurality of inner pockets 4 is configured to receive an insulin pump or any other medical device. In various embodiments, various combinations of the aforementioned pocket configurations may be comprised in any desired configuration. FIG. 5 shows an example of a user wearing the garment of the present invention with a medical device placed within one of the plurality of front inner pockets 40.

In the preferred embodiment, the plurality of front inner pockets 40 comprises a right front inner pocket 41 and a left front inner pocket 42. The right front inner pocket 41 and the left front inner pocket 42 are positioned laterally opposite each other about a lateral centerline 9 of the waist portion 1. The lateral centerline 9 coincides with the longitudinal direction. Similarly, the plurality of rear inner pockets 46 comprises a right rear inner pocket 47 and a left rear inner pocket 48, and the right rear inner pocket 47 and the left rear inner pocket 48 are positioned laterally opposite each other about the lateral centerline 9. Thus, having left and right front and rear pockets, the present invention may facilitate concealing a medical device in any position about the user's body, whether the operation was on the front, rear, left or right sides of the user's body.

Furthermore, in the preferred embodiment of the present invention, the plurality of outer pockets 5 comprises a right front outer pocket 51, a left front outer pocket 52, a right rear outer pocket 53, and a left rear outer pocket 54. The right front outer pocket 51 and the left front outer pocket 52 are positioned laterally opposite each other about the lateral centerline 9, and similarly the right rear outer pocket 53 and the left rear outer pocket 54 are positioned opposite each other about the lateral centerline 9. The right front outer pocket 51 and the right rear outer pocket 53 are positioned longitudinally opposite each other on the waist portion 1, and similarly the left front outer pocket 52 and the left rear outer pocket 54 are positioned longitudinally opposite each other on the waist portion 1. Thus, in the preferred embodiment, in addition to the inner pockets for receiving the medical device, the user is provided with typical pants pockets for carrying various items such as a wallet, cell phone, keys, or other items.

Furthermore, in the preferred embodiment, each of the plurality of front inner pockets 40 comprises at least one tube hole 43. Post-operative medical devices such as, but not limited to, JP drains and insulin pumps require tubing to be attached between the device and the user's body. Thus, while such post-operative devices are received by one of the inner pockets, at least one tube hole 43 receives the tube of the device for convenience and comfort of the user. Each of the at least one tube hole 43 traverses into the interior cavity 8 for each of the plurality of front inner pockets 40. In one embodiment, the at least one tube hole 43 comprises a first tube hole 44 and a second tube hole 45 for each of the plurality of front inner pockets 40. The first tube hole 44 and the second tube hole 45 are laterally separated from each other on each of the plurality of front inner pockets 40. This lateral side by side configuration for the first tube hole 44 and the second tube hole 45 allows for variations in the specific medical device to be received, so that the tube of the medical device may easily be inserted through a hole out of the front pocket regardless of to which lateral side of the medical device the tube is connected.

In one embodiment, the present invention takes the form of a pair of pants, also known as trousers or britches. Thus, in one embodiment the lower body portion 2 comprises a first pant leg 21 and a second pant leg 22, which are connected to the waist portion 1 in a manner typical of pants.

One additional feature desired to be included in embodiments where the lower body portion 2 is a pants portion is the ability to open the lower end of the pant legs in order to allow easy access inside the pant legs. Patients, medical providers and/or caretakers can undo the pant legs with ease in order to easily assess or perform maintenance on their condition. Additionally, the convenience of the pant leg openings allows medical personnel and the patient's caregivers to do so themselves, obviating the need of the patient to completely the pants entirely. Users with a cast or casts, braces, straps, external fixation devices, hinged braces, all knee supports and braces, or a myriad of any other lower body extremity devices can simply open up one or both pant legs with extreme ease and comfort to assess or perform maintenance on their lower body devices or conditions. Thus, the garment of the present invention allows extreme accessibility, convenience, privacy and comfort for any and all of its patients and wearers as well as medical providers and caregivers.

In one embodiment, the first pant leg 21 and the second pant leg 22 each comprise a lower leg portion 23 and at least one clothing fastener. The lower leg portion 23 is positioned opposite the waist portion 1 along the lower body portion 2. Preferably, the at least one clothing fastener comprises a plurality of zippers 24, though it is contemplated that any clothing fastener may be utilized, including, but not limited to, button snaps, hook and loop tape, magnetic strips, cloth loops, or other clothing fasteners.

The plurality of zippers 24 is interiorly integrated into the lower leg portion 23 in such a manner that each of the plurality of zippers 24 is concealed within the lower leg portion 23. This configuration may be referred to as an “invisible zipper” configuration, as the rows of interdigitating teeth are attached within the pant legs, thus being obscured from external view. Preferably, each of the plurality of zippers 24 is positioned where a seam would typically appear on a standard pair of pants. Thus, each of the invisible zippers simply resemble a standard pants seam to an observer. In turn, the invisible zippers provide privacy, accessibility and, most importantly, convenience and comfort. In one embodiment, each of the plurality of zippers 24 is 22 inches in length so that the zippers extend just above the knee of the user. However, the length of the zippers may vary in various embodiments.

More particularly, the plurality of zippers 24 comprises a first zipper 25 and a second zipper 26 for each of the first pant leg 21 and the second pant leg 22, and the first zipper 25 and the second zipper 26 are positioned laterally opposite each other on each of the first pant leg 21 and the second pant leg 22. FIG. 5 shows an illustration of a user wearing the present invention with the first zipper 25 of the second pant leg 22 undone. As discussed, the first zipper 25 and second zipper 26 are placed where the seams would be on a typical pair of pants, essentially dividing the pant legs into a front half and a rear half. It is further contemplated that in one embodiment the at least one clothing fastener may comprise only a single zipper, though two is preferable.

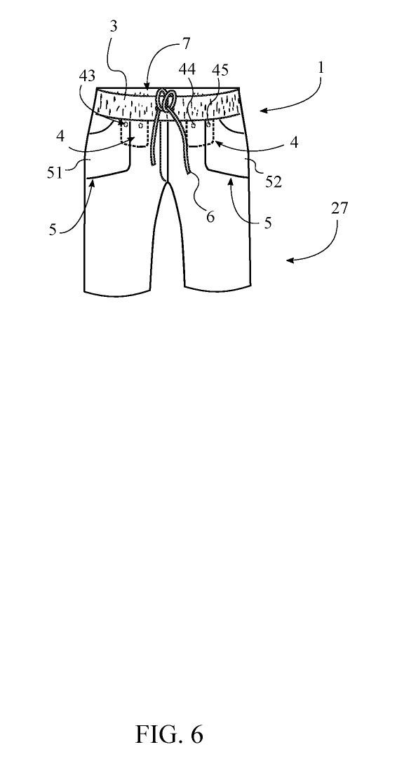

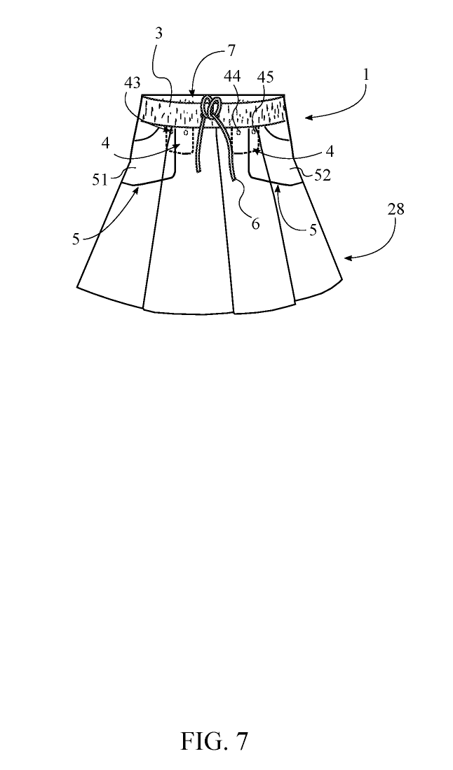

It should be noted that the present invention should not be limited to a pants configuration. The primary focus is on the inner pockets and the invisible zippers of the pant legs, and as such various lower body garment variations may be created. Thus, in one embodiment shown in FIG. 6, the lower body portion 2 comprises a shorts portion 27. In one embodiment shown in FIG. 7, the lower body portion 2 comprises a skirt portion 28. In other embodiments, the lower body portion 2 may take various other forms of lower body garments.

Therefore, in view of the foregoing disclosure, the present invention presents a post-operative and lower extremity medical device accommodating lower body garment with a plurality of inner pockets distributed around the interior of the waistband configured to receive one or more medical devices and which accommodate the tubes of the medical devices regardless of the location of the device's connection to the user's abdomen or torso, as well as invisible zippers on both pant legs for the user including amputees and patients wearing prosthetics to have the ease of access, comfort and privacy of wearing or utilizing casts, braces, straps, external fixation devices, hinged braces, all knee supports and braces, and a myriad of any other lower extremity devices, including post-operative devices.

Although the invention has been explained in relation to its preferred embodiment, it is to be understood that many other possible modifications and variations can be made without departing from the spirit and scope of the invention as hereinafter claimed.

What is claimed is:

1. A post-operative lower body garment comprises:
a waist portion;
a lower body portion;
the waist portion comprises a waistband, a plurality of inner pockets, and a plurality of outer pockets;
the lower body portion being connected to the waist portion opposite the waistband, wherein the waistband delineates an opening of the waist portion;
the plurality of inner pockets being interiorly connected within an interior cavity of the waist portion between the waistband and the lower body portion, wherein the opening traverses into the interior cavity;
the plurality of outer pockets being exteriorly connected to the waist portion between the waistband and the lower body portion;
the plurality of inner pockets comprises a plurality of front inner pockets and a plurality of rear inner pockets;
the plurality of front inner pockets and the plurality of rear inner pockets being positioned longitudinally opposite each other within the waist portion; and
each of the plurality of front inner pockets comprises at least one tube hole.

2. The invention as claimed in claim 1 comprises:
at least one of the plurality of inner pockets being configured to receive a post-operative drain reservoir.

3. The invention as claimed in claim 1 comprises:
at least one of the plurality of inner pockets being configured to receive a Jackson-Pratt drain reservoir.

4. The invention as claimed in claim 1 comprises:
at least one of the plurality of inner pockets being configured to receive an insulin pump.

5. The invention as claimed in claim 1 comprises:
each of the at least one tube hole traversing into the interior cavity for each of the plurality of front inner pockets;

6. The invention as claimed in claim 1 comprises:
the at least one tube hole comprises a first tube hole and a second tube hole for each of the plurality of front inner pockets;
the first tube hole and the second tube hole being laterally separated from each other on each of the plurality of front inner pockets;

7. The invention as claimed in claim 1 comprises:
the plurality of front inner pockets comprises a right front inner pocket and a left front inner pocket; and
the right front inner pocket and the left front inner pocket being positioned laterally opposite each other about a lateral centerline of the waist portion.

8. The invention as claimed in claim 1 comprises:
the plurality of rear inner pockets comprises a right rear inner pocket and a left rear inner pocket; and
the right rear inner pocket and the left rear inner pocket being positioned laterally opposite each other about a lateral centerline of the waist portion.

9. The invention as claimed in claim 1 comprises:
the plurality of outer pockets comprises a right front outer pocket, a left front outer pocket, a right rear outer pocket, and a left rear outer pocket;
the right front outer pocket and the left front outer pocket being positioned laterally opposite each other about a lateral centerline of the waist portion;
the right rear outer pocket and the left rear outer pocket being positioned laterally opposite each other about the lateral centerline;
the right front outer pocket and the right rear outer pocket being positioned longitudinally opposite each other on the waist portion; and
the left front outer pocket and the left rear outer pocket being positioned longitudinally opposite each other on the waist portion.

10. The invention as claimed in claim 1 comprises:
the lower body portion comprises a first pant leg and a second pant leg;
the first pant leg and the second pant leg each comprise a lower leg portion and a plurality of zippers; and
the lower leg portion positioned opposite the waist portion along the lower body portion.

11. The invention as claimed in claim 10 comprises:
the plurality of zippers being interiorly integrated into the lower leg portion, wherein each of the plurality of zippers is concealed within the lower leg portion.

12. The invention as claimed in claim 10 comprises:
the plurality of zippers comprises a first zipper and a second zipper for each of the first pant leg and the second pant leg; and
the first zipper and the second zipper being positioned laterally opposite each other on each of the first pant leg and the second pant leg.

13. The invention as claimed in claim 1 comprises:
the waistband being made of elastic material.

14. The invention as claimed in claim 1 comprises:
the waistband comprises a drawstring; and
the drawstring being positioned within the waistband, wherein the drawstring is configured to close the opening.

15. The invention as claimed in claim 1 comprises:
the lower body portion comprises a shorts portion.

16. The invention as claimed in claim 1 comprises:
the lower body portion comprises a skirt portion.
